(19) United States
(12) Patent Application Publication
(10) Pub. No.: US 2008/0166361 A1
(43) Pub. Date: Jul. 10, 2008

(54) Neutron Capture Therapy

(76) Inventor: Bipin Chandra Mujibhia Patel, Guilford (GB)

Correspondence Address: CHOATE, HALL & STEWART LLP, TWO INTERNATIONAL PLACE, BOSTON, MA, 02110, US

(21) Appl. No.: 11/950,908

(22) Filed: Dec. 5, 2007

Related U.S. Application Data
(63) Continuation of application No. 10/478,589, filed on May 4, 2004, now abandoned.

(30) Foreign Application Priority Data
May 25, 2001 (GB) 0112844.6

(51) Int. Cl. A61K 39/44; A61P 43/00

(52) U.S. Cl. 424/178.1

(57) ABSTRACT

The present invention relates to immunoconjugates for use in neutron capture therapy, in particular Boron neutron capture therapy, for killing target cells such as tumours. The immunoconjugate of the invention comprises a monoclonal antibody having an affinity of at least 10¹¹ l/mol and a cross-reactivity of less than 10%, and a neutron capture agent. More preferably, the affinity of the monoclonal antibody is at least 10¹² l/mol.

The present invention relates to the use of neutron capture therapy (NCT) in killing target cells such as tumours. In particular, the present invention relates to improved means and methods for targeting neutron capture agents, such as boron, to cells and for delivering ionising radiation to the cells. The means and methods of the present invention enable improved targeting of cells and optimisation of neutron capture therapy for clinical use.

Neutron capture therapy is a bi-modal anti-cancer therapy which is based upon the ability of the stable and non-radioactive nucleus of the neutron capture agent, typically ¹⁰Boron isotope, to absorb neutrons and undergo a fission reaction, producing ionising radiation. Its ability to absorb neutrons is several thousand times greater than that of elements which constitute living tissues, such as hydrogen, oxygen and carbon, making it a suitable agent for neutron capture therapy. The fission reaction is summarised by the following equation:

¹⁰B+¹nₜₕ=⁷Li+αparticle(⁴He)+2.4 MeV

In this nuclear reaction, the ¹⁰Boron nucleus absorbs a neutron and forms a metastable nuclide intermediate, ¹¹Boron, which then spontaneously disintegrates to form a ⁴He particle and a ⁷Li particle. These particles have a combined energy of 2.4 MeV, and produce ionisation tracks having a range of about one cell diameter, i.e. 7 to 9 μm. As a result, the reaction is capable of limited destruction of boron containing cells. The effectiveness of NCT as a cancer therapy therefore relies on the ability to produce a relatively large radiation dose in the tumour compared with the surrounding, healthy tissue. This is achieved by preferential accumulation of the neutron capture agent in the tumour compared to healthy tissue.

In conventional radiotherapy, the biological effect of the therapy is spread over the entire irradiated area, and a high radiation dose is required to generate the destructive ionisation tracks to produce the desired biological effect. This is a limiting factor in the effectiveness of conventional radiotherapy. NCT, in contrast, allows in situ selectivity by targeting of the boron isotope, and employs high energy, short range particles which demonstrate a greater destructive potential than the conventional low energy radiation beams used. As a result, lower doses of NCT are required to produce the same therapeutic effect as conventional therapy. In addition, because NCT is not dependent upon the oxygen levels of the targeted cells, it can provide effective therapy where the tumour is anatomically compromised and therefore hypoxic.

NCT can be used to treat a variety of cancers which are normally treated with radiotherapy, including for example lymphomas and skin cancers, as well as cancers of the breast, lung, head and neck, bone, prostate, pancreas and cervix NCT may also be used in combination with surgery to help shrink the size of the tumour and reduce normal tissue loss. In contrast to conventional radiotherapy which is administered up to 30 times in a six week period, NCT can be administered over a period of 2 to 4 days, and as a result is less demanding for the patient.

To date, the most popular neutron capture agent used has comprised ¹⁰Boron, the reaction thus becoming known as Boron Neutron Capture Therapy (BNCT). To reap the benefits of BNCT, the boron atoms must be selectively localised in the target tissue. One of the major challenges in the development of BNCT has been the identification of suitable ways of selectively targeting the boron atoms to the tissue to be irradiated. In addition, it is desirable to use agents which will not only target boron to this tissue, but also enable the boron atoms to persist in the target tissue for a sufficient length of time such that the radiation delivered will be effective and selective for these cells (Soloway et al (Chem Rev 98 1515-1562 (1998)).

Early boron capture compounds used in the 1950s and 1960s were chosen on the basis of localisation studies, and included compounds such as sodium borate and boric acid. Whilst these compounds showed low toxicity and adequate tumour concentrations, they had tumour:brain differentials which were transient and reached unity within a short space of time. Later compounds such as p-carboxyphenylboronic acid (PCPB) and sodium decahydrodecaborate showed promise in terms of toxicity and tissue selectivity, but became highly concentrated in the blood, resulting in radiation injury leading to morbidity and mortality of patients.

Further work tried to address the problem of high boron concentrations in the blood, and led to the identification of second generation boron compounds, mercaptoundecahydrododecaborate (BSH) (Soloway et al., J. Med. Chem 10 714 (1967) the contents of which are incorporated herein by reference) and 4-dihydroxyborylphenylalanine (BPA). Whilst BSH has been used extensively in clinical trials, the basis upon which its tumour accretion ability is achieved is not understood. In addition, optimal parameters for the safe and effective use of BNCT with compounds such as BSH have yet to be identified.

The studies described above have led to the conclusion that for NCT to be successful as a therapy and maintain the above mentioned advantages over conventional radiotherapy, several criteria must be met. Firstly, the neutron capture agent such as the ¹⁰Boron isotope must be present in sufficient quantities at the target site. Secondly, it must show selectivity for the tissue site of interest over normal tissue, and preferably show a tumour:healthy tissue ratio of greater than 1, and preferably between 3 and 5. Lack of specificity in targeting and low tumour:healthy tissue ratios have severely hindered the development of NCT as an effective therapy. Thirdly, the tumour-blood ratio of the neutron capture agent must not be less than one. Finally, the neutron capture agent should not be toxic to the patient.

Soloway et al postulate that these criteria may be achieved by the use of several agents, each with the capacity to target different tumour receptors or organelles such as the nucleus, mitochondria and golgi apparatus. However, the vast majority of compounds used to date do not internalise into the cell, but remain outside of the cell in the intercellular space. In addition, the toxicity level of many of these compounds renders them unsuitable for clinical BNCT.

Whilst the above mentioned compounds are considered to have some potential as commercial BNCT compounds, work has continued to find compounds having better tumour specificity, as a result of different biochemical and physiological mechanisms, than that of existing boron compounds. These third generation boron compounds include nucleic acid precursors, amino acids, lipoproteins, DNA binders, antigen binders, and other compounds such as porphyrins and phthalocyanines, radiation sensitisers and amines.

The potential for antibodies as tumour targeting boron carriers was suggested early on in BNCT research, and initial studies focused on the compounds which may be attached to the antibodies. Preferred compounds for this purpose included polyhedralboranes and stable carboranes, due to their ability to hold high numbers of boron atoms. In order to achieve a sufficient level of boron loading, it was estimated that each antibody molecule would have to undergo 20 to 100 discrete chemical reactions, whilst avoiding significant alteration to its conformation and targeting ability. The initial conclusion from this research was that inserting such small molecules into an antibody was not productive, and it would be preferable to attach to the antibody one or several macromolecules, each carrying 200-1000 boron atoms (Soloway et al).

Immunoconjugates comprising macromolecules such as polylysine and aminodextran have been developed. For example, European Patent No 0 294 392 (Shih et al) describes conjugates in which the boron addend is covalently bound to aminodextran, which in turn is covalently bound to an amino group of the carbohydrate portion of the antibody by a reduced Schiff base linkage. In a similar vein, U.S. Pat. No. 5,443,953 (Hansen et al) describes the use of antibody fragments having an polymeric boron carrier attached to a carbohydrate moiety of the variable light chain, thus reducing the need to use a complete antibody. Whilst these antibodies and fragments thereof maintain sufficient immunoreactivity for use in BNCT, in vivo only a small proportion of the immunoconjugate administered actually reaches the target cells, with the remainder accumulating in the liver. Studies have shown that the larger the molecular weight of the boron carrier, the greater the propensity of the immunoconjugate to accumulate in the liver (Barth et al., Bioconjugate Chem. 5 58 (1994)). In addition, the use of large polymers as boron carriers has the disadvantage that the chain lengths of the polymers is inconsistent and only a range of molecular weights can be created. Such a cocktail mixture is generally not considered to be a good drug candidate.

As a result of these limitations in the use of immunoconjugates, different approaches have been used for macromolecular targeting. For example, U.S. Pat. No. 5,856,741 (Griffiths et al) describes a method for targeting boron to tumour cells by administering a conjugate of streptavidin bound to an antibody, which is selective for an antigen of a tumour cell, and a boron containing compound conjugated to biotin, such that biotin/strepatvidin binding causes boron to accumulate at the tumour cells. Typically, these systems use BSH as the boron containing compound for attachment to streptavidin (Soloway et al). As the system does not load the antibody itself with macromolecules, the pre-targeting system avoids the problems of accumulation of the boronated antibody in the liver. The antibodies suggested for use by Griffiths et al have about 60% specificity for the antigen, and a cross-reactivity of approximately 35%. These parameters would, however, result in excess amounts of boron in healthy cells.

A second example is U.S. Pat. No. 5,851,527 (Hansen et al) which describes a method for enhancing the targeting capabilities of an antibody, for example for use in BNCT. The method comprises first administering an antibody conjugated to an enzyme, and then administering a substrate-agent conjugate, such that the enzyme transforms the substrate-agent into a product agent which localises at the tumour site for effective treatment. In particular, the system described suggests the use of carboranes as boron carriers, attached to amino-dextrans as the substrate-agent. Whilst the idea of local boron release as a means of increasing the amount of boron delivered is a good idea in principle, its complex nature has meant that there are very few systems which exist in practice. In particular, it is likely that the carborane, such as BSH, will be cleaved from the substrate by non-specific enzymes, and the boron isotope will end up in, normal healthy tissues, thus decreasing the specificity required for effective BNCT.

Polymers which have conventionally been attached to antibodies to secure effective delivery of the right amount of boron to the target site are usually in the form of cocktail mixtures of polymers, as it is usually impossible to manufacture a consistent batch of polymers of a single molecular weight. This distribution again affects the efficiency and parameters of the BNCT system.

U.S. Pat. No. 5,612,017 (Muira et al) describes iodinated sulfidohydroboranes, for example Na₄B₁₂I₁₁SSB₁₂I₁₁ (BSH) and IBSSB which are useful in BNCT but also allow visualisation of the compound for purposes of rapidly and directly targeting the tumour and estimating the neutron dose. U.S. Pat. No. 5,877,165 (Muira et al) describes non-toxic boronated porphyrins for use in BNCT. Porphyrins show selectivity for tumours, persistent uptake, and internalisation in target cells, making them a potential candidate for use in BNCT. By introducing boron cages into the porphyrin nucleus, a higher concentration of boron can be obtained.

To further increase the efficiency of NCT, which is essentially a bimodal system, each component of the system can be manipulated independently of the other. The present inventors have observed that the biological outcome of NCT is the function of several variables, including the quality of the neutron source, the concentration of the neutron capture agent at the target cells, the degree of internalisation of the neutron capture agent into the target cells, and the homogeneity distribution within the tumour. Whilst researchers in the field of NCT have considered each of the above factors, no one to date has examined the interplay between these variables, and the effect which this may have on NCT as a viable therapeutic system. As a direct result, the parameters of NCT, including the amount of neutron capture agent required, the means of targeting and the provision of neutron irradiation, have been grossly misaligned with those actually necessary for the delivery of an optimal therapeutic system. In particular, mathematical modeling to date has estimated that for efficient BNCT in vivo, 15-35 μg ¹⁰Boron per g of tissue is required, using a neutron beam of 10⁹ neutrons per second per cm². This equates to approximately 10⁹ boron atoms per target cell. The present inventors have realised that a total possible antigen occupancy would be in the range of 10³ to 10⁵ antigens per cell (on the basis that normally only 1 to 10% occupancy of the 10⁴ to 10⁶ antigens can be achieved). This level of occupancy was not considered by Griffiths et al and the prior art systems are not efficient enough to overcome the low occupancy and provide the amount of boron required for effective BNCT, according to the above estimates. In addition, a neutron flux of 10⁹ neutrons per second per cm² is too high, and will cause more damage through unwanted radiation than therapeutic benefit.

U.S. Pat. No. 5,630,786 suggests the use of a neutron beam in which at least 85% of the neutrons have an energy level of above 100 keV. By definition, 100 keV neutrons are not fast or epithermal neutrons, but are of an energy level which generate highly damaging knock-on protons. For this reason, neutrons of this energy level are not biologically acceptable and are therefore not suitable for BNCT. Rather, it has been suggested that the neutron energy level should lie between 0.1 eV and 10 keV, with an ideal of 0.4 eV (Nigg, Idaho MEL report, US Government 1999).

To date nuclear reactor beams have been the only source of neutrons for BNCT

These neutron beams typically contain 30-40% of contaminants such as fast neutrons, and α and β radiation. These contaminants contribute to the damage of normal tissues and thereby reduce the effectiveness of NCT.

It is apparent from the above that despite the potential of NCT as a cancer therapy, optimal parameters for its clinical use need to be identified if the benefits of the therapy are to outweigh the detrimental side effects. The present invention aims to overcome or ameliorate problems associated with known NCT systems, by providing for the first time parameters which allow safe and effective therapy.

In a first aspect of the invention there is provided an immunoconjugate comprising (i) a monoclonal antibody capable of binding to an antigen with an affinity constant of at least 10¹¹ l/mol and having a cross reactivity of less than 10%; and (ii) a neutron capture agent.

In a second aspect of the invention, there is provided an immunoconjugate comprising (i) a monoclonal antibody capable of binding to an antigen with an affinity constant of at least 10¹¹ l/mol and having a cross reactivity of less than 10%; and (ii) a neutron capture agent, for use in neutron capture therapy. The second aspect also provides the immunoconjugate of the second aspect for cell ablation including treatment of cancer by neutron capture therapy.

In a third aspect of the invention, there is provided the use of an immunoconjugate comprising (i) a monoclonal antibody capable of binding to an antigen with an affinity constant of at least 10¹¹ l/mol and having a cross reactivity of less than 10%, and (ii) a neutron capture agent, in the manufacture of a pharmaceutical composition for use in neutron capture therapy. The third aspect also provides the use of an immunoconjugate of the third aspect in the manufacture of a pharmaceutical composition for use in cell ablation including the treatment of cancer by neutron capture therapy.

In a fourth aspect of the present invention, there is provided a method of cell ablation, preferably by neutron capture therapy, the method comprising administering to a subject an immunoconjugate comprising (i) a monoclonal antibody capable of binding to an antigen with an affinity constant of at least 10¹¹ l/mol and having a cross reactivity of less than 10%; and (ii) a neutron capture agent; and a supply of neutrons. The method of cell ablation includes the treatment of cancer.

The present invention is not restricted to the treatment of cancer by NCT, but may be used in any method which requires NCT for tissue ablation. For the purposes of this invention, the term “cell ablation” also includes tissue ablation. Examples of conditions which may benefit from NCT include non-malignant diseases, non-metastatic benign tumours, uterine fibroids, arthritis, breast adenoma, menohrragina, benign prostate hyperplasia and destruction of selective cardiac structures or clots. NCT may also be used where tissue ablation is preferable to procedures such as surgery, photodynamic therapy, cryosurgery, thermal laser ablation and vaporisation.

The present invention overcomes problems associated with known methods for NCT, and for the first time provides a practical system for the administration of NCT without the risk of adverse side effects such as toxic poisoning and damage to healthy tissue. This is achieved by the use of antibodies having an affinity constant of at least 10¹¹ l/mol. Such high affinity antibodies have not been previously suggested for use in NCT.

The present invention has enabled the parameters for BNCT to be redefined. Mathematical models based upon early results using contaminated neutron beams and non-specific antibodies suggested that 10⁹ boron atoms were required per cell for ablation. Despite the ongoing work in the field of BNCT, these parameters have never been reconsidered, and treatments have realistically failed due to the inability to provide this level of ¹⁰Boron. The use of the high affinity antibodies of the invention has shown that in fact a lesser amount of boron is necessary for effective cell kill i.e. only 10⁴-10⁷ boron atoms per cell are required. The use of very high affinity antibodies in the invention has reduced the estimated amount of boron required for cell kill, and as a result has addressed many of the problems which hindered early previous BNCT systems. For example, as less neutron capture agent is required, direct antibody loading is possible and more effective. This reduces the risk of adversely affecting the specificity of the antibody and as a result, the specificity affinity and retention time of the immunoconjugates are improved and any loss due to inactivation upon conjugation of the neutron capture agent can be tolerated. The problems of toxicity are also ameliorated, and compounds previously thought to be too toxic may now have utility in NCT if administered in smaller amounts.

The present invention is also advantageous over antibody systems involving two or three step processes, because it for the first time enables effective direct load of the antibody.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023, the contents of which are incorporated herein by reference.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, B. S. et al., Nature 341:544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (vi) bispecific single chain Fv dimers PCT/US92/09965) and (ix) “diabodies”, multivalent or multispecific fragments constructed by gene fusion (WO94/13804; P. Hollinger et al., Proc. Natl. Acad. Sci. USA 90:6444-6448 (1993)).

An “antigen binding domain” is the part of an antibody which comprises the area which specifically binds to and is complementary to part or all of an antigen or target site. Where an antigen or target site is large, an antibody may only bind to a particular part of the antigen, which part is termed an epitope. An antigen binding domain may be provided by one or more antibody variable domains. Preferably, an antigen binding domain comprises an antibody light chain variable region (VL) and an antibody heavy chain variable region (VH).

The antibodies used in the invention are monoclonal antibodies, and may be derived from any mammal, for example murine or ovine. Preferably, the antibodies are derived from large mammals, most preferably sheep. The antibodies may be immunoglobulins of any class, for example IgG, IgM, IgA, IgD, or IgE, or hybrid antibodies with dual or multiple antigen specificity's. Advantages of fragments over whole antibodies include faster clearance from the body of unbound antibodies from the body, better penetration and reduced immunogenicity.

Preferably, the antibodies of the present invention are humanised, and comprise at least the hypervariable region from a monoclonal antibody having an affinity of at least 10¹¹ l/mol and a cross reactivity of less than 10%, and part of the constant region or variable region from a human immunoglobulin. Such antibodies may be produced according to methods known in the art, for example EP 0 574 461 (the contents of which are incorporated herein by reference).

It is preferred if the antibody of the present invention comprises the CDR of the high affinity monoclonal antibody of the invention. By “high affinity” is meant an antibody having a binding affinity of at least 10¹¹ l/mol and preferably a cross reactivity of less than 10% in a human antibody framework. The antibody may further comprise one or more of the other CDRs of the heavy or light chains of the high affinity antibody, and may comprise the hypervariable region. The variable region other than the hypervariable region may be derived from the variable region of a human antibody. Methods for making such antibodies are known in the art, for example, in Winter, U.S. Pat. No. 5,225,539, the contents of which are incorporated herein by reference.

The variable region of the antibody outside of the hypervariable region may also be derived from monoclonal antibody. Methods for making such antibodies are known in the alt, including, for example, those described in U.S. Pat. Nos. 4,816,397 by Boss (Celltech) and 4,816,567 by Cabilly (Genentech), the contents of which are incorporated herein by reference. Thus, in a preferred embodiment, the antibody comprises the human antibody framework and a substantial portion of the variable region of the high affinity antibody.

The human antibody framework is preferably all or a part of the constant region of a human antibody. For example, humanised antibodies based on all or a part of the VL region of the high affinity antibody may be attached at their C-terminal end to antibody light chain constant domains including human Ck or Cl chains. Similarly, antibodies based on all or a part of the VH region of the high affinity antibody may be attached at their C-terminal end to all or part of an immunoglobulin heavy chain derived from any antibody isotype, e.g. IgG, IgA, IgE and IgM and any of the isotype sub-classes, particularly IgG1 and IgG4. IgG1 is preferred.

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associated with each other to form an antigen binding site: antigen binding sites are formed by the association of the first domain of one polypeptide within the multimer with the second domain of another polypeptide within the multimer (WO94/13804), the contents of which are incorporated herein by reference.

Where bispecific antibodies are to be used, these may be conventional bispecific antibodies, which can be manufactured in a variety of ways (Hollinger & Winter, Current Opinion Biotechnol. 1993 4:46-449), the contents of which are incorporated herein by reference, e.g. prepared chemically or from hybrid hybridomas, or may be any of the bispecific antibody fragments mentioned above. It may be preferable to use scFv dimers or diabodies rather than whole antibodies. Diabodies and scFv can be constructed without an Fc region, using only variable domains, potentially reducing the effects of anti-idiotypic reaction. Other forms of bispecific antibodies include the single chain “Janusins” described in Traunecker et al., EMBO Journal 10:3655-3659 (1991).

Bispecific diabodies, as opposed to bispecific whole antibodies, may be readily constructed and expressed in E. coli. Diabodies (and many other polypeptides such as antibody fragments) of appropriate binding specificities can be readily selected using phage display (WO94/13804), the contents of which are incorporated herein by reference from libraries. If one arm of the diabody is to be kept constant, for instance, with a specificity directed against antigen X, then a library can be made where the other arm is varied and an antibody of appropriate specificity selected.

A substantial portion of an antibody variable domain will comprise at least the three CDR regions, together with their intervening framework regions. Preferably, the portion will also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of high affinity antibodies made by recombinant DNA techniques may result in the introduction of N- or C-terminal residues encoded by linkers introduced to facilitate cloning or other manipulation steps, including the introduction of linkers to join variable domains of the invention to further protein sequences including immunoglobulin heavy chains, other variable domains (for example in the production of diabodies) or protein labels.

The VH or VL binding domains of the high affinity antibody may be used to screen for complementary domains capable of forming a two-domain specific binding member which has in vivo properties as good as or equal to the antibodies disclosed herein. This may be achieved by phage display screening methods using the so-called hierarchical dual combinatorial approach as disclosed in WO92/01047 (the contents of which are incorporated herein by reference), in which an individual colony containing either an H or L chain clone is used to infect a complete library of clones encoding the other chain (L or H) and the resulting two-chain specific binding member is selected in accordance with phage display techniques such as those described in that reference.

The antibodies used in the present invention have an affinity for the antigen of the target cell above 10¹¹ l/mol. More preferably, the affinity constant is 10¹² l/mol. The affinity constant is a measure of the strength of binding between the antibody and antigen, and is the concentration of the bound form divided by the product of the concentrations of the unbound form at equilibrium. Methods for measuring the concentrations are available to persons skilled in the art. As dissociation of the antibody-antigen conjugate will be favoured over association if the affinity constant falls below a threshold value, the affinity constant is critical to ensure that the antibody-antigen conjugate remains stable and at a high dose at the target site. Antibodies having an affinity above 10¹¹ l/mol show improved localisation at the target site, and are thus being suitable for the most challenging of applications. They also have a long half life, thus allowing longer periods between dosing and because a lower dose is needed compared to conventional antibodies, show reduced immunogenicity and increased cost effectiveness. Methods for obtaining the antibodies of the affinity of the present invention will be known to persons skilled in the art, although it is preferred that the antibodies are raised in sheep, and identified, using the methods described in international patent application No. WO 00/12556. The high affinity antibodies of WO 00/12256 are “acid resistant” antibodies which favour association over disassociation and therefore have larger retention time at the target site. This results in a higher affinity for the antigen. The “acid resistant” antibodies may be produced by methods known in the art, for example using hybridoma technology comprising the fusion of B-lymphocytes from an immunised animal (preferably sheep) with a suitable fusion partner. The “acid resistant” monoclonal antibodies can be identified in an acid-washed enzyme-linked immunosorbent assay (EIA), which comprises the steps of:
(i) incubating first and second samples of the antibody in microtitre plate wells coated with the target antigen at a concentration within the linear response pail of a standard curve at pH7.2 for 1 hour at 37° C.;
(ii) removing unbound antibody from both wells;
(iii) incubating the first sample with PBS at pH 7.2 for 1 hour at 37° C.;
(iv) incubating the second sample at pH 3 or below for 1 hour at 37° C.;
(v) removing unbound antibody from both samples;
(vi) incubating the first and second samples with anti-antibody alkaline phosphate conjugate for 1 hour at 37° C.;
(vii) removing any unbound conjugate from both samples;
(viii) adding PNPP substrate to the samples, measuring the absorbance at 450 nm, and determining the amount of antibody bound to antigen, wherein for an acid resistant antibody the amount bound in the second sample is >50% of the amount bound in the first sample.

Preferably, the antibodies used in the invention have low cross reactivity with other antigens, for example antigens of normal tissue cells. By cross reactivity is meant the degree to which the antibody binds to other antigens as well as to the antigen it has been raised against. For example, a cross reactivity of 50% means that half of the antibody binding will be to antigens other than the specified target antigen. Preferably, the cross reactivity of the antibodies used in the invention will be not more than 10%, more preferably not more than 5% and most preferably not more than 2%. The cross reactivity of an antibody can be measured using methods available to persons skilled in the art, as described in international patent application No. WO00/12556.

Immunoconjugates of the invention are molecules comprising an antibody as defined above, and a neutron capture agent. Preferably, immunoconjugate retains the immunoreactivity of the antibody, meaning that the antibody has the same, or only slightly reduced ability to bind to the antigen after conjugation to a neutron capture agent compared to before conjugation.

In an alternative aspect of the present invention, there are provided conjugates comprising a high affinity binding agent and a neutron capture agent. Preferably, the binding agent also has low cross reactivity with non-target sites, and more preferably has an affinity for the target site of at least 10⁻¹¹/mol, more preferably 10⁻¹²/mol. The cross reactivity of the binding molecule is preferably less than 10%. Examples of suitable binding molecules include ligands, proteins, protein fragments, lipoproteins, hormones, charged molecules, oligonucleotide memetics, peptide memetics, polypeptides, mono-, oligo- or poly-saccharides, vitamins, lectins and low molecular weight ligands such as folate. Preferably, the binding molecule will be capable of crossing the vasculature of a tumour or tissue to be ablated. Other embodiments of this aspect are as for the other aspects, mutatis mutandis. In addition, the all references herein to immunoconjugates shall include the conjugates of this aspect, and all references to antibodies shall include the binding molecules of this aspect.

The target sites for the antibodies of the invention may include tumours, infectious and parasitic lesions, antigens, fibrin clots, myocardial infarctions, atherosclerotic plaques, damaged cells, non-cancerous cells and lymphocytic autoimmune cells. Preferably, the antibody is capable of binding to an antigen on the surface of a target site which is to be the subject of NCT treatment, for example a tumour cell or a normal cell to be ablated. Preferably, the antigen is specific to the target site, such that the antibody will preferentially bind to a site which is to be the subject of treatment, with minimal binding to other sites such as to “normal” or “healthy” tissue, which for the purpose of the present invention means tissue which is not to be ablated by NCT treatment. Preferably, where the antigen is on the surface of a cell, one which is capable of internalisation, so that the neutron capture agent is delivered to the inside of the cell. Examples of suitable antigens for targeting for internalisation are CEA and EGFr. Alternatively, the antigen may be one specific to organelles of a target cell such as the golgi apparatus, endothelial reticulum, mitochondria and nuclear membrane. This is achieved by targeting the antibody to an antigen which is present on the surface of the cell, and then internalised, usually to one of the above organelles. Such antigens are recycled, usually every 4 to 12 or 24 to 48 hours.

By the term “neutron capture agent” is meant both a neutron capture element which is directly conjugated to the antibody of the invention, or carriers comprising neutron capture elements. The neutron capture element is preferably one which is able to absorb neutrons and undergo a fission reaction. Preferred elements are those which are non-radioactive, have a stable isotope, are able to capture thermal neutrons and produce energetic particles and energy having a limited range. Examples of suitable neutron capture elements include ⁶Li, ²²Na, ¹⁰B, ²²CO, ¹¹³CO, ¹²⁶I, ¹³⁵Xe, ¹⁴⁸Pm, ¹⁴⁹Sm, ¹⁵¹Eu, ¹⁵⁵Gd, ¹⁵⁷Gd, ¹⁶⁴Dy, ¹⁸⁴Os, ¹⁹⁹Hg, ²³⁰Pa, ²³⁵U and ²⁴¹Pu. As ¹⁰Boron satisfies all of the above criteria, it is the most preferred neutron capture element.

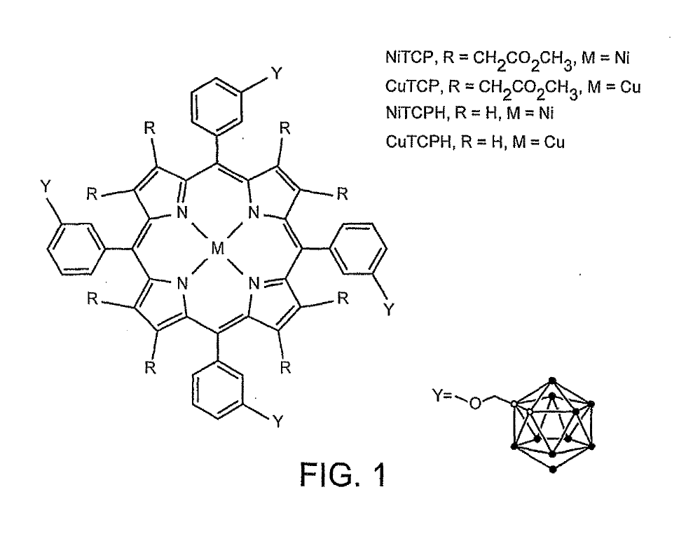

Preferred neutron capture agents are in the form of a carrier comprising a neutron capture element as defined above. Preferred carriers are those which are able to cross the vasculature of a tumour, and may be identified using standard methods using in vivo animal models and radioactively labeled carriers. Most preferred carriers are those having low toxicity and a high targeting capability for target sites without the need for a targeting system such as an antibody. Such carriers are typically small, and preferably have a molecular weight of less than 800,000 Da, more preferably less than 50,000 Da, most preferably between 10 and 30,000 Da. Where the neutron capture agent is boron, the preferred boron carriers include carborane cages (Solway et al), halogenated sulfidohydroboranes (U.S. Pat. Nos. 5,455,022, 5,612,017 and 5,653,957, the contents of which are incorporated herein by reference), porphyrins (U.S. Pat. No. 4,959,356, the contents of which are incorporated herein by reference), boronated porphyrins (U.S. Pat. No. 5,877,165, the contents of which are incorporated herein by reference), small polymer chains, and dendromers and complex polymers such as starburst molecules (Soloway et al, the contents of which are incorporated herein by reference). Most preferred are the lipophilic carboranyltetraphenylporphyrins (LTCP's), which include NiTCP, CuTCP, NiTCPH and CuTCPH or halogenated derivatives where the 6 hydrogens are replaced by a halogen namely by one or more of P, Cl, Br, I or a combination thereof. These latter compounds are preferred because they do not cause thrombocytopenia at doses above 200-400 μg/g body weight. They also have an unusually low toxicity, and are able to deliver more boron to target sites than other porphyrins such as 2,4-bis[a,b-dihydroxyethyl]-dentro-porphyrin (BOPP) or 2,4-divinyl-nido-o-carboranyl-deuteo-porphyrin (VCDP). A further advantage of the LCTP's is that their production can be scaled up more readily than other porphyrins, thus making them a stronger candidate for the basis of a large scale pharmaceutical preparation. Of the LCTP's, CuTCPH is the most preferred as it has the most desirable biodistribution and toxicological properties, and shows minor skin photosensitization. In an alternative embodiment of the invention, the metal ion of the LTCP's may be replaced by gadolinium, gold, technetium, germanium, vanadium, manganese, iron, ruthenium, zinc, technetium, chromium, platinum, cobalt, nickel, copper, indium, gadolinium, tin, yttrium, gold, barium and tungsten. The most preferred metals are copper and nickel. Preferred boron carriers are shown in FIG. 1.

Other carriers include Fab fragments, PEG polymers, liposomes and macromolecules which have the ability to cross the vasculature of a tumour or tissue to be ablated (Seymour et al, Eur. J. Cancer 31A (5) 766-770 (1995)).

Whilst Soloway et al suggest that it would be advantageous for the ¹⁰Boron to be internalised into the target cell, to date this has not been achieved with known systems. It has always been thought that the exact location of the ¹⁰Boron is critical to achieving efficient cell kill without damaging normal tissue, and to maximise efficient use of BNCT reaction, the ¹⁰Boron must be as close to the cellular organelles as possible. To overcome the problem of lack of internalisation, previously proposed antibody systems, such as those of Griffiths et al which place the ¹⁰Boron further away from the cellular organelles, have used a high concentration of ¹⁰Boron. As a result, such systems have encountered problems with toxicity and immunogenicity. The preferred carriers of the invention are low in toxicity, and in combination with the high affinity antibodies of the invention, can for the first time be administered in concentrations which are sufficiently high to be of clinical use without the need for internalisation. Advantageously, however, the immunoconjugates of the invention are capable of internalisation, and thus enable a low toxicity, highly efficient NCT system for the first time.

The novel combination of the highly specific small molecule carriers, and the very high affinity monoclonal antibodies provides an efficient targeting system having a combined efficiency which is greater than that of either component alone.

The neutron capture agents are preferably attached directly to the antibody. In some cases, however, it may be preferred to use indirect attachment. Suitable methods will be known to persons skilled in the art. The neutron capture agent may be attached to the antibody to produce an immunoconjugate using standard conjugation chemistry, for example as described in Childs et alt (J. Nuc. Med. 26:293 (1985), the contents of which are incorporated herein by reference).

As mentioned above, to achieve efficient cell kill, the inventors have shown that, for boron, only 10⁴-10^{6 10}Boron atoms are required per cell. The exact number of neutron capture atoms required is a function of the cross sectional area of the element. Boron has a cross sectional area of 3838 barn (where 1 barn=10⁻²⁴ cm²), whilst Xe has a cross sectional area of 2600000 barn, ¹¹³Co has a cross sectional area of 1900 barn and Gd has a cross sectional area of 225000 barn. The higher the cross sectional area, the less neutron capture element is required because the probability of neutron capture is increased. The number of neutron capture element atoms required is a function of the elements' cross section divided by 3838, taking into account the energy of the fission particles produced and their energy range for causing cell damage. The cross sectional area of the neutron capture elements will be known to persons skilled in the art.

The delivery of this amount of neutron capture element, or boron, to a target site will depend on various factors, such as the affinity of the immunoconjugate for the antigen, the capacity of the carrier, the nature of the target antigen and its' capacity for internalisation of the immunoconjugate into the target cell. Table 2 shows the typical number of boron atoms held by each type of boron carrier, and the number of antibodies required, per cell to achieve various levels of boron in the cells to be ablated (based upon an antigen occupancy of 10⁴ in man).

TABLE 2

| | | | | Nucleus and |
|---|---|---|---|---|
| | | Cell membrane | Cytoplasm | Nuclear membrane |
| | | No. Carriers per | No. Carriers per | No. Carriers per |
| | | antibody, to | antibody, to | antibody, to achieve |
| | No. Boron atoms | achieve 10⁸-10⁹ | achieve 10⁷-10⁸ | 10⁵-10⁶ |
| Boron carriers | per carrier | boron atoms | boron atoms | boron atoms |
| Carboranecages | 9-12 | 100-1000 | 10-100 | 1-10 |
| LCTP's | 100 | 10-100 | 1-10 | 1 |
| Oligomers/star- | 200-1000 | 1-10 | 1 | 1 |
| burst molecules | | | | |

The dosage of the immunoconjugate will vary depending upon factors such as the patient's age, weight, height, sex, medical condition and previous medical history, as well as factors such as the location and severity of condition to be treated, and the degree and rate of deposit of the antibody and boron conjugate at the target site, the degree and rate of internalisation of the conjugate, and the rate of clearance of the conjugate or boron. Typically, it is desirable to provide the recipient with a dosage of immunoconjugate which is capable of delivering 10⁴ to 10⁷ atoms of neutron capture element, preferably ¹⁰Boron, per cell, which equates to 15-25 μg/g tissue. Using these parameters, the skilled person will be able to calculate the exact dosage required, using standard methods known in the art. In some cases, however, a higher or lower dose may be administered. In some cases, it may be desirable to administer the immunoconjugate in stages, or by slow release, rather than in a single dose. This may be advantageous, for example, where the number of immunoconjugates to be administered is greater than the maximum number of antigens which may be occupied. Where the antigens are capable of internalisation, the administration of the immunoconjugate may be staggered to coincide with “re-appearance” of the antigen. The suitable time intervals will depend on factors such as cell and antigen type, and may be calculated by persons skilled in the art. The antibodies of the invention preferably have a retention time of 1 to 2 days, and more preferably 4 to 6 days, thus allowing a longer window for treatment and fractionation of the radiation doses. For slow release preparations, the immunoconjugate may be implanted at or close to the tumour site or in the dermis, from where it will be carried in the vasculature to the tumour site. Where the antibody of the immunoconjugate is specific for a non-internalising antigen on the surface of the target cell, it may be desirable to follow administration of the immunoconjugate with a non-conjugated boron carrier, such as one mentioned above.

The dose of immunoconjugate administered to a recipient must result in a therapeutically acceptable amount, i.e. its presence must be sufficient to result in cell damage or ablation upon administration of a neutron supply.

The immunoconjugates can be formulated according to known methods to produce pharmaceutically useful compositions, whereby the immunoconjugates are combined in a mixture with a pharmaceutically acceptable carrier. The resulting composition is pharmaceutically acceptable if it can be tolerated by the recipient. Examples of suitable carriers will be known to persons skilled in the art, and are described in Remingtons' Pharmaceutical Sciences (Maack Publishing Co., Easton Pa.).

In the fourth aspect of the invention, there is supplied to the recipient of the immunoconjugate a supply of neutrons, which react with the boron atoms of the immunoconjugate to produce an alpha particle which is capable of ablating the cell. The neutron supply is preferably in the form of monochromatic neutrons, which are free from contaminants such as fast neutrons, and gamma, beta radiation or X-rays. The use of clean neutrons free from such contaminants ensures a superior biological outcome compared to the results obtained with conventional, contaminated neutron beams. The monochromatic neutrons used in the present invention may be obtained from various sources, such as the fusionstar device of Sved et al (Am. Inst. Phys 1-56396-825/99 (1999) the contents of which are incorporated herein by reference, and available from Daimler-Chrysler Aerospace). Preferably, the neutrons emitted from these sources have an energy level of between 0.01 eV and 0.5 eV, and most preferably 0.1 eV and 0.01 eV.

In a method of the fourth aspect, the neutrons are preferably supplied as a beam, which has a cross section larger that the tissue to be ablated. Typically, the beam will have a cross section such that it is capable of capturing tissue slightly outside the tissue to be ablated, i.e. having a margin of between 0.5 mm to 10 mm. The beam is preferably adjusted according to the size and location of the tissue to be ablated, and the exact cross section of the beam may be calculated according to these variables by persons skilled in the art. In practice, after administration of the immunoconjugate, sufficient time is allowed to elapse to enable clearance of the immunoconjugate from normal tissue, in order to minimise the damage to such tissue. Clearance times will of course depend on various factors and can be easily calculated by persons skilled in the art. At the appropriate time, the patient is positioned in front of the neutron beam, so that the target tissue is within the neutron irradiation field. The physical parameters of the beam and the boron distribution within the patient will ensure that the radiation exposure of the target tissue is maximised, and that of the normal or healthy tissue is minimised.

The preferred embodiments of each aspect apply to the other aspects, mutatis mutandis.

FIG. 1 shows the structure of various LCTP compounds, for use as boron carriers.

The present invention will now be described with reference to the following non-limiting example:

### EXAMPLE

### Antibody/Fab Fragment Conjugation

High affinity antibodies are raised for a number of tumour models. These include glioma (rat 9 L gliosarcoma), colon cancer (mouse), melanoma (murine Harding Passey melanoma), breast cancer (murine EMT-6 adenocarcinoma) and prostate cancer. The antibodies and Fab fragments are purified as described in the International Patent Application WO00/12556 for conjugation with a sulphurhydrial containing boron compound, for example boronated polylysin (BPL), BSH or others listed below. Such —SH containing boron carrying molecules can be reacted with an maleimido group (B. Arch et al Hybridoma 5. S43 (1986)) represented via heterobifunctional reagents.

### Example (i)

### Boronated Poly-DL-Lysine (BPL)

BPL was attached to the antibody and the Fab fragments using N-succinimidyl 3-(2-pyridyldithio)propionate (SPDP) and m-maleimidobenzoyl N-hydroxysulfosuccinimide ester (MBS). Masked sulphurhydrial groups were introduced into BPL by the addition of SPDP to generate SPDP-BPL. The sulphurhydrial groups were then unmasked using dithiothreitol (DTT) resulting in the reactive compound BPL-SH.

In a separate reaction with the antibody or the Fab fragment maleimido groups were incorporated by adding MBS to the protein solution (physiological saline) at 4 degrees centrigrade (or room temperature) to generate antibody-MB (equivalent to 1 to 1 mole reaction or 10-1000 or over to 1 mole of protein to provide a large number of attachment sites). The fewer the MBS attachments is preferable, so as to conserve antibody function.

The BPL-SH and the antibody-MB are then linked by the addition of sulphurhydrial group on BPL to the double bond on the maleimido group of the antibody, to generate the Antibody-NH—S—BPL macromolecule.

The number of attachments of the BPL is controlled via the manipulation of the molar reactions. The antibody-BPL or the Fab-BPL were administered in a physiological saline preparation. See Soloway et al (supra) Scheme 6 p 1544, the contents of which are incorporated herein by reference.

### Example (ii)

BSH (B₁₂H₁₁SH²): As above, the BSH was directly reacted to the antibody-MBS or the Fab-MBS and was prepared for administration in a physiological saline.

### Example (iii)

Cs₂B₁₂H₁₁—SS(CH₂)₂COO[N(CO)₂(CH₂)₂] was also used as above to attach to the antibody and Fab fragments to produce a water soluble macromolecule.

When Boron content was measured using direct coupled plasma atomic emission spectrometry (Coderre et al., Int. J. Radiat. Oncol. Biol. Phys. 30: 643-652 (1994) the contents of which are incorporated herein by reference) these protein-Boron compounds were found to contain between 100 to over 2500 ¹⁰B atoms per protein. In some instances over 3,500 ¹⁰B atoms were found to be attached to the protein.

### Example (iv)

Similarly the “starburst” dendrimer (Soloway et al (supra) FIG. 49 p 154) consisting of polyamido amino groups arranged in a starburst pattern displayed 12 reactive amino groups and was reacted with carborane Na(CH₃)NB₁₀NCO to produce a boronated starburst derimer. Reactions were carried out such that not all of the NH2 reactive sites were allowed to react with the antibody-MBA sites. This generated an antibody-starburst molecule that was prepared for administration in a physiological saline.

### Example (v)

The boronated porphyrin CuTCPH (Muira et al., U.S. Pat. No. 5,612,017, the contents of which are incorporated herein by reference) with an SH group was used as above to prepare a CuTCPH antibody and CuTCPH Fab fragment.

### Animal Numbers and Species

Studies using rodents involved standard, established techniques and statistical methods for data analysis. On average, groups of 8 rats per data point were used in pharmacokinetic, biodistribution and radiobiological studies. The animal models were male Fischer 344 rats (250-300 g), female BALB mice (20-25 g) and SCID mice (20-25 g). In all studies, anaesthesia was maintained with ketamine (120 mg/kg) and xylazine (20 mg/kg). Animals were monitored on a daily basis for general health. Animals were euthanised, as required, under anaesthesia.

### Boron Pharmacokinetics, Biodistribution and Toxicology

Evaluation of new boronated antibodies took place using appropriate tumour models. These include glioma (rat 9 L gliosarcoma), colon cancer (mouse), melanoma (murine Harding Passey melanoma), breast cancer (murine EMT-6 adenocarcinoma) and prostate cancer (prostate xenograft in SCID mice), all transplanted subcutaneously. Pharmacokinetic/biodistribution analysis' involved the administration of graded doses of a boronated antibody, with sampling (blood, tumour, normal tissues) at time intervals of up to 6 days after administration. Antibodies delivering >30 μg boron/g to tumour with a tumour:blood boron partition ratio of >3:1 were considered suitable for further evaluation. Studies also involved observations on behaviour, changes in body weight, chemical and haematological tests (Miura et al., Brit. J. Radiol. 847: 773-781 (1998)).

The boron content of relevant normal tissues (skin, oral mucosa, brain, spinal cord and blood) and implanted tumours were measured using direct coupled plasma atomic emission spectrometry (Coderre et al, supra).

### Radiobiology Studies—Neutron Irradiation's

Standard experimental collimators, irradiation jigs and body shielding were used for irradiation's. Standard dosimetric procedures are well established and using gold foils, thermoluminescent dosimeters and ionisation chambers (Coderre et al., Radiat. Res. 129: 290-296 (1992); Morris et al., Radiother. Oncol. 32: 248-255 (1994) and Morris, et al Radiother. Oncol. 32: 144-153 (1994) the contents of which are incorporated herein by reference). The irradiation's were cared out on the thermal neutron beam at the MIT Medical Research Reactor Facility in Boston, USA.

### Pre-Clinical Therapeutic Studies

Experimental evaluation of therapeutic efficacy entailed localised irradiation of tumour-bearing animals with graded doses of thermal neutrons in the presence of the boronated antibody. The administration protocols and the timing after administration at which irradiation begins were determined by prior pharmacokinetic and biodistribution studies. The assessment consisted of quantification of changes in tumour volume, incidence of tumour ablation and long-term survival. By way of comparison, and to enable calculation of RBE/CBE factors, graded doses of x-rays and of the thermal beam alone was given.

### Histology

Standard histopathological analysis was carried out on irradiated normal tissues and tumours using standard techniques. Animals were perfusion-fixed using 10% buffered formal saline.

### Data Analysis

Dose-response data using the various endpoints was assessed using probit analysis, and ED50 and TCP50 values were derived from the fitted curves. These values were used to calculate RBE/CBE factor values. Appropriate control groups were used in all studies. In therapy experiments, the percentage survival versus time following irradiation was analysed using the Kaplan-Meier method. The survival times in each of the experimental groups was ordered and ranked for comparison by a non-parametric statistical technique, Wilcoxin 2-Sample Test.

1. An immunoconjugate for targeting a neutron capture agent to a target cell comprising (i) a monoclonal antibody capable of binding to an antigen of the target cell with an affinity constant of at least 10¹¹ l/mol and having a cross reactivity of less than 10%; and (ii) a neutron capture agent comprising a lipophilic carrier.

2. An immunoconjugate according to claim 1, wherein the monoclonal antibody is ovine derived.

3. An immunoconjugate according to claims 2, wherein the monoclonal antibody is acid resistant.

4. An immunoconjugate according to claim 1, wherein the antibody comprises the hypervariable region from an antibody capable of binding to an antigen with an affinity constant of at least 10¹¹ l/mol and having a cross reactivity of less than 11%, and a constant region from a human antibody.

5. An immunoconjugate according to claim 1, wherein the neutron capture agent comprises a neutron capture element which is ¹⁰Boron.

6. (canceled)

7. An immunoconjugate according to claim 5, wherein the lipophilic carrier is a carborane cage, a halogenated sulphidohydroborane, or a porphyrin.

8. An immunoconjugate according to claim 7 wherein the 1 carrier is a lipophilic carboranyl tetraphenylporphyrin.

9-11. (canceled)

12. A method of cell ablation comprising administering to a subject an immunoconjugate according to claim 1; and a supply of neutrons.

13. A method according to claim 12 wherein the immunoconjugate is in the form of a pharmaceutical composition.

14. A method according to claim 13 wherein the pharmaceutical composition is suitable for slow release of the immunoconjugate.

15. A method according to claim 12 wherein the supply of neutrons is in the form of monochromatic neutrons.

16. A method according to claim 12 wherein the neutrons have an energy range of 0.01 eV to 0.5 eV.

17. A method according to claim 12, wherein the monoclonal antibody is ovine derived.

18. A method according to claims 17, wherein the monoclonal antibody is acid resistant.

19. A method according to claim 12, wherein the antibody comprises the hypervariable region from an antibody capable of binding to an antigen with an affinity constant of at least 10¹¹ l/mol and having a cross reactivity of less than 10%, and a constant region from a human antibody.

20. A method according to claim 12, wherein the neutron capture agent comprises a neutron capture element which is ¹⁰Boron.

21. A method according to claim 20, wherein the neutron capture agent further comprises a carrier.

22. A method according to claim 21 wherein the carrier is a carborane cage, a halogenated sulphidohydroborane, or a porphyrin.

23. A method according to claim 22 wherein the carrier is a lipophilic carboranyl tetraphenylporphyrin.

24. A pharmaceutical composition comprising a pharmaceutically acceptable carrier and an immunoconjugate of claim 1.

25. A pharmaceutical composition according to claim 24, wherein the monoclonal antibody is ovine derived.

26. A pharmaceutical composition according to claims 25, wherein the monoclonal antibody is acid resistant.

27. A pharmaceutical composition according to claim 24, wherein the antibody comprises the hypervariable region from an antibody capable of binding to an antigen with an affinity constant of at least 10¹¹ l/mol and having a cross reactivity of less than 10%, and a constant region from a human antibody.

28. A pharmaceutical composition according to claim 24, wherein the neutron capture agent comprises a neutron capture element which is ¹⁰Boron.

29. A pharmaceutical composition according to claim 28, wherein the lipophilic carrier is a carborane cage, a halogenated sulphidohydroborane, or a porphyrin.

30. A pharmaceutical composition according to claim 29, wherein the lipophilic carrier is a lipophilic carboranyl tetraphenylporphyrin.
